(19) United States
(12) Patent Application Publication
(10) Pub. No.: US 2025/0248677 A1
(43) Pub. Date: Aug. 7, 2025

(54) X-RAY CT APPARATUS

(71) Applicant: FUJIFILM Corporation, Tokyo (JP)

(72) Inventors: Akie NAKAYAMA, Tokyo (JP); Hiroki SADAMARU, Tokyo (JP)

(73) Assignee: FUJIFILM Corporation, Tokyo (JP)

(21) Appl. No.: 19/023,288

(22) Filed: Jan. 16, 2025

(30) Foreign Application Priority Data
Feb. 6, 2024 (JP) 2024-016325

(51) Int. Cl. A61B 6/00; A61B 6/03

(52) U.S. Cl. CPC A61B 6/56; A61B 6/035

(57) ABSTRACT

Provided is an X-ray CT apparatus that can collect an abrasion powder during a rotation of a rotation frame. The X-ray CT apparatus includes a fixed frame, a rotation frame that is rotatable with respect to the fixed frame and includes an X-ray source and an X-ray detector, a slip ring that rotates in synchronization with the rotation frame, a power supply brush and a signal transmission brush that are in contact with the slip ring, a cover that covers the slip ring, the power supply brush, and the signal transmission brush, and a collection container that collects an abrasion powder. The collection container is provided in the cover, and has an opening portion positioned below the power supply brush in a direction of gravitational force and extends to a side of a forward direction of an air flow occurring due to a rotation of the slip ring.

### CROSS-REFERENCE TO RELATED APPLICATION

The present application claims priority under 35 U.S.C § 119(a) to Japanese Patent Application No. 2024-016325 filed on Feb. 6, 2024, which is hereby expressly incorporated by reference, in its entirety, into the present application.

### BACKGROUND OF THE INVENTION

### 1. Field of the Invention

The present invention relates to an X-ray CT apparatus, and more particularly, to an X-ray CT apparatus comprising a brush and a slip ring.

### 2. Description of the Related Art

In general, the X-ray CT apparatus has a slip ring unit as a unit that performs power supply and signal transmission between a fixed frame and a continuously rotating rotation frame. The slip ring unit includes a plurality of the slip rings that rotate together with the rotation frame, and the brush that is in contact with the slip ring.

In general, the brush is in sliding contact with the slip ring in order to maintain electrical connection. Therefore, in a case where the slip ring rotates, a conductive abrasion powder occurs due to the sliding contact between the slip ring and the brush.

JP2018-027239A discloses an X-ray CT apparatus comprising a scattering prevention cover for an abrasion powder, a collection container disposed below a rotation frame, and a collection container disposed below a brush in order to deal with an occurrence of the abrasion powder. The collection container below the brush collects the falling abrasion powder.

Meanwhile, the collection container below the rotation frame collects the abrasion powder guided downward along the cover in a case where a rotation is stopped.

### SUMMARY OF THE INVENTION

The X-ray CT apparatus comprises two types of brushes, a power supply brush and a signal transmission brush. The power supply brush has a larger contact area with the slip ring than the signal transmission brush. That is, a size of the abrasion powder from the power supply brush is larger than a size of the abrasion powder from the signal transmission brush. Therefore, it is necessary to collect two types of abrasion powders having different sizes in the X-ray CT apparatus.

In addition, since the abrasion powder may come out of a gap even in a case where a cover for preventing the abrasion powder from scattering is attached, it is preferable to induce and collect the abrasion powder during a rotation of the rotation frame. In a case where the abrasion powder comes out of the cover, the abrasion powder is scattered inside and outside the apparatus, and a wide range of cleaning is required. In addition, cooling efficiency is decreased because the abrasion powder accumulates in a filter of a cooling fan of the apparatus.

The present invention has been made in order to solve the above-described problems, and an object thereof is to obtain an X-ray CT apparatus capable of collecting an abrasion powder during a rotation of a rotation frame.

An X-ray CT apparatus of a first aspect comprises a fixed frame, a rotation frame that is rotatable with respect to the fixed frame and includes an X-ray source and an X-ray detector, a slip ring that rotates in synchronization with the rotation frame, a power supply brush and a signal transmission brush that are in contact with the slip ring, a cover that covers the slip ring, the power supply brush, and the signal transmission brush, and a collection container which is provided in the cover and has an opening portion and collects an abrasion powder and in which the opening portion is positioned below the power supply brush in a direction of gravitational force and extends to a side of a forward direction of an air flow occurring due to a rotation of the slip ring.

In the X-ray CT apparatus of a second aspect, the opening portion is spread toward the cover.

In the X-ray CT apparatus of a third aspect, the collection container extends in a tangential direction with respect to an outer periphery of the cover.

In the X-ray CT apparatus of a fourth aspect, a porous member that is disposed inside the collection container is provided.

In the X-ray CT apparatus of a fifth aspect, the power supply brush is positioned below an imaginary horizontal line passing through a rotation axis of the rotation frame in the direction of gravitational force.

In the X-ray CT apparatus of a sixth aspect, the power supply brush and the signal transmission brush are disposed at different positions in a circumferential direction of the slip ring.

In the X-ray CT apparatus of a seventh aspect, the power supply brush and the signal transmission brush are disposed at the same position in a circumferential direction of the slip ring.

In the X-ray CT apparatus of an eighth aspect, the signal transmission brush is disposed on an upstream side of the power supply brush in a rotation direction of the slip ring.

In the X-ray CT apparatus of a ninth aspect, the cover is disposed on a side opposite to the fixed frame with respect to the slip ring.

According to the present invention, it is possible to collect an abrasion powder during a rotation of a rotation frame.

### BRIEF DESCRIPTION OF THE DRAWINGS

FIG. 1 is a perspective view for describing an appearance of an entire X-ray CT apparatus.

FIG. 2 is a front view of a rotary disk portion of the X-ray CT apparatus with a gantry cover removed.

FIG. 3 is a front view of a state where a rotation frame is detached from the rotary disk portion of FIG. 2.

FIG. 4 is a sectional view taken along line 4-4 of the rotary disk portion of FIG. 2.

FIG. 5 is a view schematically showing a slip ring and a brush which are disposed in a gantry.

FIG. 6 is a sectional view taken along line 6-6 of FIG. 5.

FIG. 7 is a sectional view taken along line 7-7 of FIG. 5.

FIG. 8 is an enlarged view of a collection container of a first embodiment.

FIG. 9 is a view for describing a modification example.

FIG. 10 is a view for describing a second embodiment.

FIG. 11 is a sectional view taken along line 11-11 of FIG. 10.

### DESCRIPTION OF THE PREFERRED EMBODIMENTS

### First Embodiment

An X-ray computed tomography (CT) apparatus according to an embodiment of the present invention will be described.

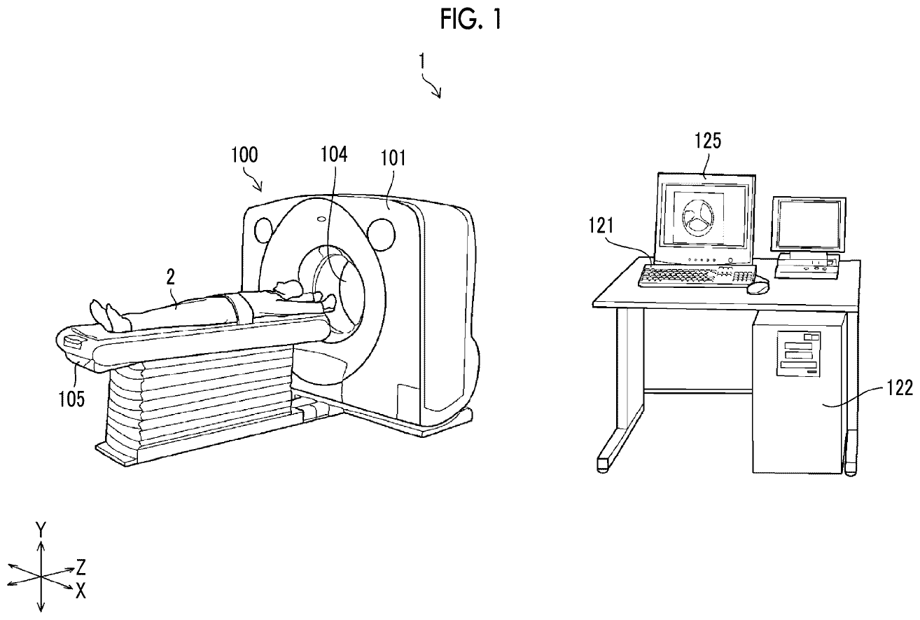

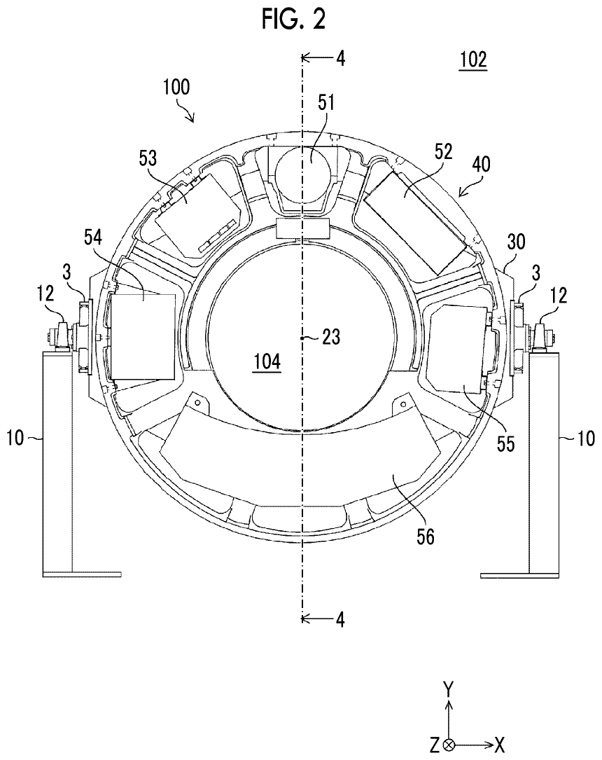

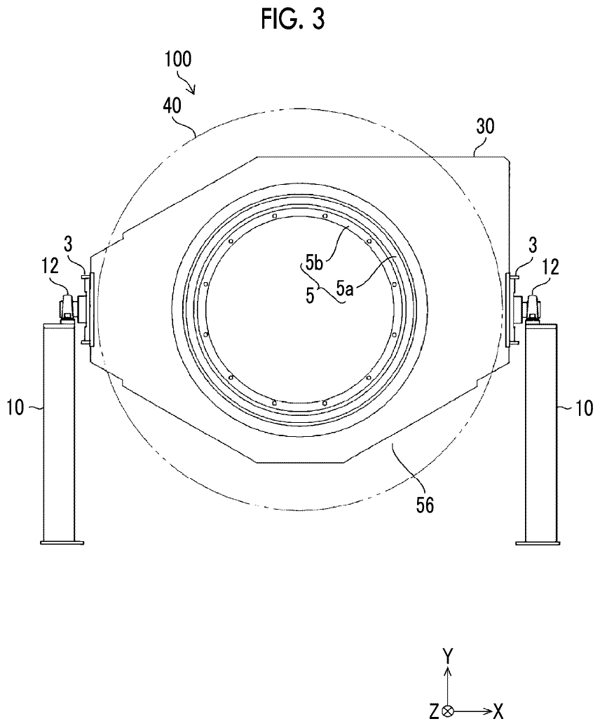

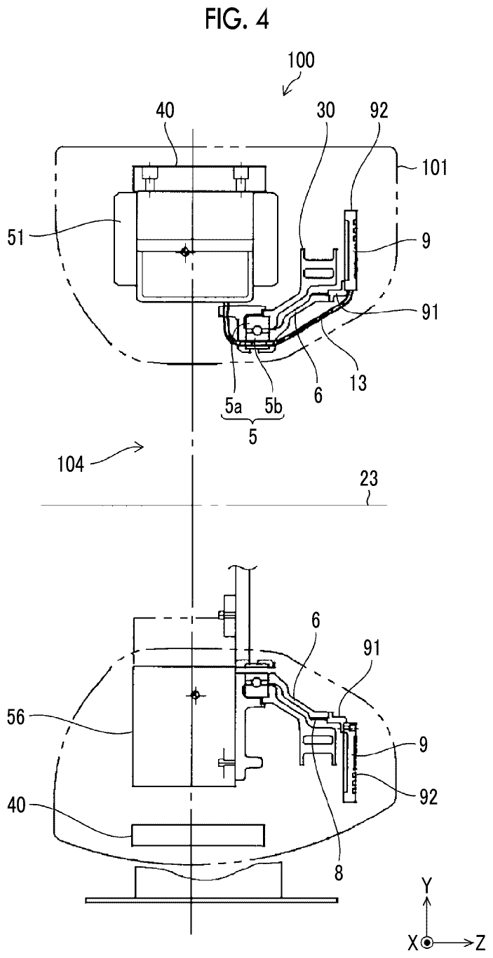

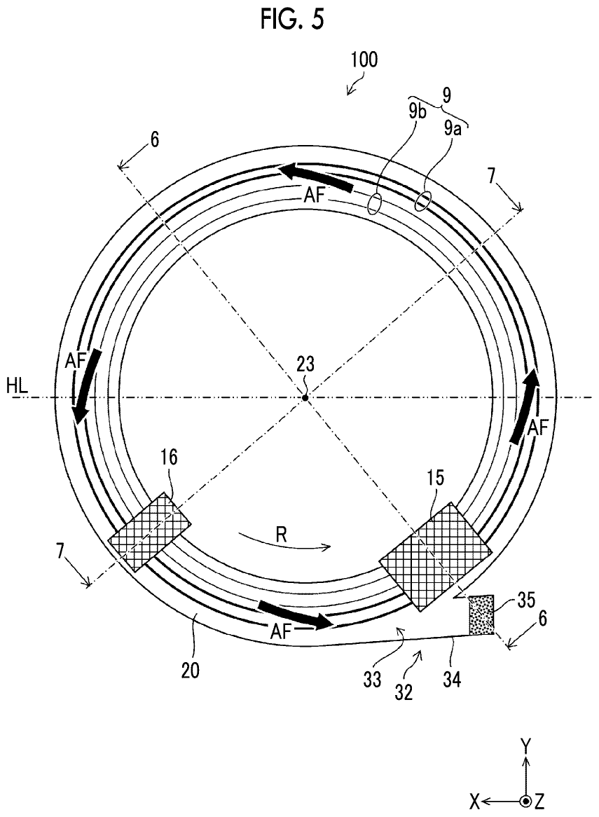

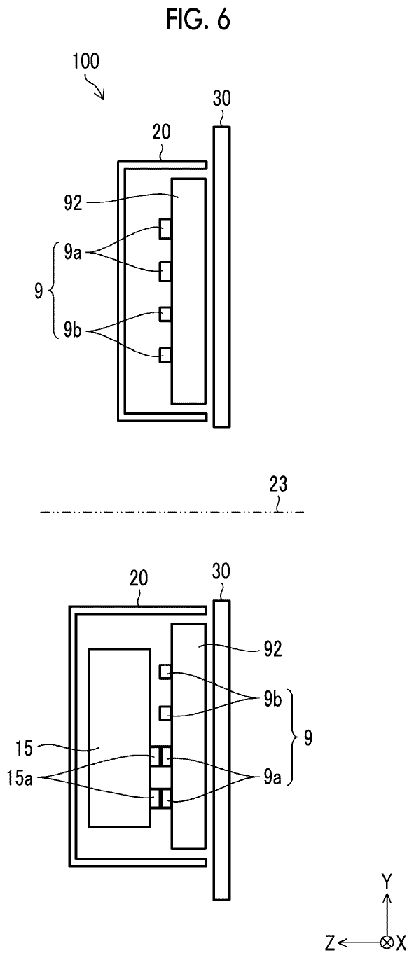

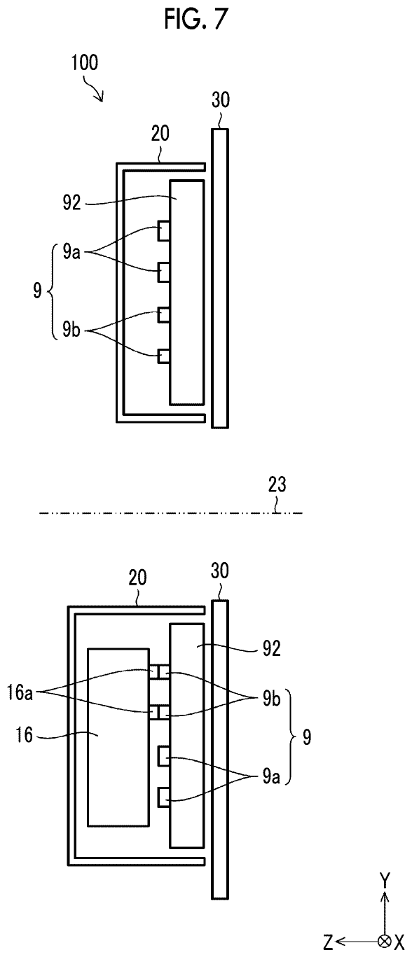

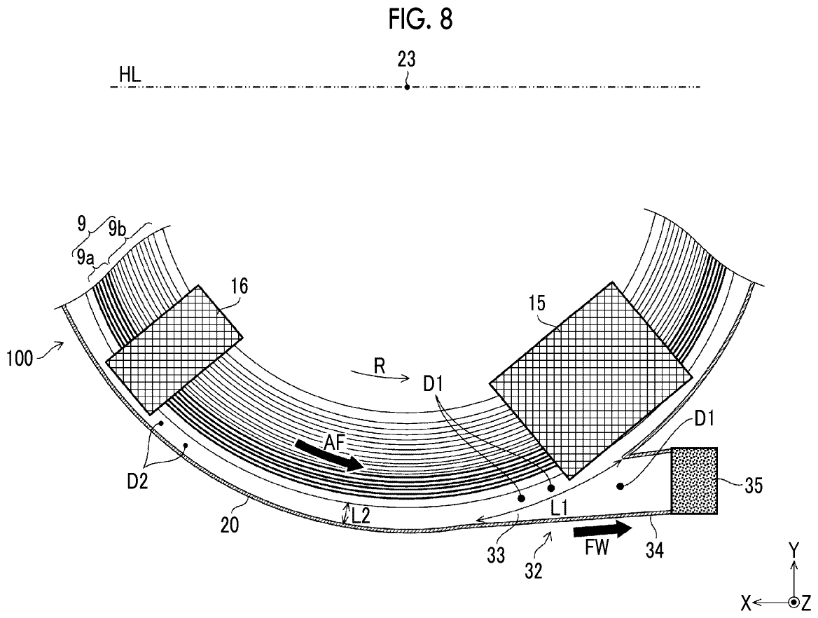

FIGS. 1 to 8 show a structure of the X-ray CT apparatus of the present embodiment. FIG. 1 is an external perspective view of the X-ray CT apparatus, FIG. 2 is a front view of a rotary disk portion with a cover of a gantry removed, and FIG. 3 is a front view of a structure with a rotation frame detached from the rotary disk portion. FIG. 4 is a sectional view taken along line 4-4 of the rotary disk portion. FIG. 5 is a view schematically showing a slip ring and a brush which are disposed in a gantry 100 of the X-ray CT apparatus. FIG. 6 is a sectional view taken along line 6-6 of FIG. 5. FIG. 7 is a sectional view taken along line 7-7 of FIG. 5. FIG. 8 is an enlarged view of a collection container of an embodiment.

Note that a three-dimensional coordinate system illustrated in FIG. 1 illustrates an example of definition of directions in an X-ray CT apparatus 1. The X axis, the Y axis, and the Z axis of the three-dimensional coordinate system are examples and are not limited thereto. For ease of understanding in the following description, the X axis, the Y axis, and the Z axis in the X-ray CT apparatus 1 are defined in the same direction in any drawing. The Z-axis direction is a body axis direction of a subject. The Y-axis direction is an up-down direction of the subject, and is a direction parallel to a direction of gravitational force. The X-axis direction is a left-right direction of the subject, and is a horizontal direction orthogonal to the direction of gravitational force.

The X-ray CT apparatus 1 comprises the gantry 100, a bed 105, an input device 121, an image calculation device 122, and a display device 125.

The gantry 100 includes a gantry cover 101 and a rotary disk portion 102 (refer to FIG. 2 and the like) disposed inside the gantry cover 101. The rotary disk portion 102 comprises a rotation frame 40 provided with an opening portion 104, a fixed frame 30 that supports the rotation frame 40 from a rear side via a bearing 5, a pulley 6 for rotationally driving the rotation frame 40, and a tilt mechanism that tiltably supports the fixed frame 30. The tilt mechanism includes a stand 10, a side frame 3 fixed to the fixed frame 30, and a bearing 12 that tiltably supports the side frame 3 on the stand 10. The fixed frame 30 is an example of a fixed frame according to the embodiment of the present invention. The rotation frame 40 is an example of the rotation frame according to the embodiment of the present invention.

An X-ray tube 51, an X-ray detection unit 56, a cooling device 52, a controller 55, a high-voltage generation units 54 and 53, and the like are mounted on the rotation frame 40. The rotation frame 40 is rotated around a subject 2. The X-ray detection unit 56 is disposed to face the X-ray tube 51. The X-ray detection unit 56 detects X-rays transmitted through the subject 2 to measure a spatial distribution of the transmitted X-rays. The X-ray tube 51 is an example of an X-ray source according to the embodiment of the present invention. The X-ray detection unit 56 is an example of an X-ray detector according to the embodiment of the present invention.

The image calculation device 122 performs CT image reconstruction by performing calculation processing on measurement data of the X-ray detection unit 56. The display device 125 displays a CT image created by the image calculation device 122. The input device 121 accepts an input of an imaging condition or the like from an operator.

As shown in FIGS. 3 and 4, the fixed frame 30 is disposed on a rear side of the rotation frame 40 of the rotary disk portion 102. The fixed frame 30 has, for example, a polygonal shape in a front view. An end part of the fixed frame 30 on a rotation frame 40 side is connected to an outer ring 5a which is a fixing portion side of the bearing 5, and supports the bearing 5.

The conical pulley 6 is disposed inside the opening portion 104 of the fixed frame 30, and an end part of the pulley 6 on the rotation frame 40 side is connected to an inner ring 5b of the bearing 5 on a rotation portion side. The inner ring 5b of the bearing 5 is connected to the rotation frame 40.

A motor (not shown) is disposed, and a driving belt 8 is wound between the motor and the pulley 6. The rotation frame 40 is rotationally driven by rotating the pulley 6 via the motor.

A plate-shaped and ring-shaped holding member 92 is attached to a distal end (opening end) of the pulley 6 via a connection tool 91. A slip ring 9 composed of a plurality of conductors is disposed in the holding member 92. The principal plane of the holding member 92 on which the slip ring 9 is disposed is orthogonal to a rotation axis 23 of the rotation frame 40. Although an example in which the slip ring 9 is disposed in the holding member 92 attached to the rotation frame 40 has been described, the slip ring 9 can be disposed in the rotation frame 40.

A plurality of cables 13 are each connected to the slip ring 9. The plurality of cables 13 pass through the inner side of the pulley 6 and reach the rotation frame 40 positioned on a front side of the bearing 5.

As shown in FIG. 5, a plurality of conductor rings constituting the slip ring 9 have different diameters from each other and are arranged concentrically with respect to the rotation axis 23 on the principal plane of the holding member 92.

A power supply brush device 15 and a signal transmission brush device 16 are disposed at positions facing the slip ring 9. The power supply brush device 15 and the signal transmission brush device 16 are physically spaced from each other. The power supply brush device 15 and the signal transmission brush device 16 are disposed at different positions in the circumferential direction of the slip ring 9, and the signal transmission brush device 16 is disposed on the upstream side of the power supply brush device 15 to be spaced in the circumferential direction with respect to a rotation direction R of the slip ring 9. In a case where the slip ring 9 is divided into two equal parts by a straight line passing through the reference object (power supply brush device 15) serving as a reference and through the rotation axis 23, the comparison object (signal transmission brush device 16) can be defined as upstream in a case where the comparison object is positioned on the left side in a rear view.

The slip ring 9 is composed of a power supply conductor 9a and a signal transmission conductor 9b. The power supply conductor 9a is disposed on an outer side (opposite to the rotation axis 23) of the signal transmission conductor 9b. The power supply conductor 9a is configured to be wider than the signal transmission conductor 9b. Although an example in which the power supply conductor 9a is disposed on the outer side of the signal transmission conductor 9b has been described, the power supply conductor 9a may be disposed on an inner side (side of the rotation axis 23) of the signal transmission conductor 9b.

A cover 20 for preventing scattering is disposed to surround the outer periphery of the slip ring 9. The cover 20 has a ring shape along the shape of the slip ring 9 as a whole. A collection container 32 is provided in a part of the cover 20 to collect the abrasion powder. The collection container 32 will be described below.

The power supply brush device 15 and the signal transmission brush device 16 are disposed below an imaginary horizontal line HL passing through the rotation axis 23 of the rotation frame 40 in the gantry 100, in the direction of gravitational force.

In a case where the rotation frame 40 rotates about the rotation axis 23, the slip ring 9 rotates in the rotation direction R indicated by the arrow in synchronization with the rotation frame 40. As the slip ring 9 moves in the rotation direction R, an air flow AF indicated by an arrow direction is generated.

As shown in FIG. 6, the slip ring 9 is disposed on the principal plane opposite to the fixed frame 30 with the holding member 92 interposed therebetween. The power supply brush device 15 comprises a power supply brush 15a, and the power supply brush 15a is in contact with the power supply conductor 9a of the slip ring 9.

As shown in FIG. 7, the signal transmission brush device 16 comprises a signal transmission brush 16a, and the signal transmission brush 16a is in contact with the signal transmission conductor 9b of the slip ring 9.

As shown in FIGS. 6 and 7, the power supply brush 15a has a larger contact area with the slip ring 9 than the signal transmission brush 16a, corresponding to the width of each of the power supply conductor 9a and the signal transmission conductor 9b.

In a cross-sectional view, the cover 20 for preventing scattering is disposed to cover the slip ring 9, the power supply brush device 15 (power supply brush 15a), and the signal transmission brush device 16 (signal transmission brush 16a). The cover 20 is positioned on a side opposite to the fixed frame 30 with respect to the slip ring 9.

The cover 20 is formed in a substantially U-shape with a side of the fixed frame 30 open in the cross-sectional view. The two corner portions of the cover 20 may have a rounded shape or an angular shape (for example, a right angle shape) in the cross-sectional view. Since the cover 20 covers the slip ring 9, the power supply brush device 15 (power supply brush 15a), and the signal transmission brush device 16 (signal transmission brush 16a), the conductive abrasion powder generated by the sliding contact between the slip ring 9 and the power supply brush 15a and the signal transmission brush 16a can be retained inside the cover 20.

The slip ring 9 is an example of a slip ring according to the embodiment of the present invention. The power supply brush 15a is an example of a power supply brush according to the embodiment of the present invention, and the signal transmission brush 16a is an example of a signal transmission brush according to the embodiment of the present invention. The cover 20 is an example of a cover according to the embodiment of the present invention.

As shown in FIG. 8, in the gantry 100 of the X-ray CT apparatus 1, the collection container 32 for collecting the conductive abrasion powder is provided in the cover 20. The collection container 32 comprises an opening portion 33 for receiving abrasion powders D1 and D2, and a tubular container main body 34 in which the abrasion powders D1 and D2 are accumulated.

The opening portion 33 is connected to the cover 20 and serves as a receiving port for guiding the abrasion powders D1 and D2 that fall or scatter inside the cover 20 to the container main body 34. The opening portion 33 is positioned below the power supply brush 15a in the direction of gravitational force and disposed in the cover 20. In a case where a plurality of the power supply brushes 15a are present, the opening portion 33 may be positioned below at least one power supply brush 15a in the direction of gravitational force. The opening portion 33 has a shape that is spread toward the cover 20. The spread shape means that a length L1 of the opening portion 33 is larger than a gap length L2 between the slip ring 9 and the cover 20. The shape of the opening portion 33 is not limited thereto, and various shapes can be applied.

The container main body 34 has a tubular shape having a space inside, such as a cylindrical shape or a prismatic shape, and extends from the opening portion 33 to a side of a forward direction FW of the air flow AF generated by the rotation of the slip ring 9. Here, the air flow AF is an air flow generated on the upstream side of the opening portion 33 in the rotation direction R, and the collection container 32 may extend in the forward direction FW of the air flow AF. The direction in which the collection container 32 extends and the forward direction FW of the air flow AF may be parallel to each other, and the direction in which the collection container 32 extends and the forward direction FW do not have to be parallel to each other as long as the collection container 32 can collect the abrasion powders D1 and D2 via the air flow AF. The shape and size of the container main body 34 can be changed as appropriate as long as the container main body 34 has a space for accumulating the abrasion powders D1 and D2.

It is preferable that a porous member 35 is provided inside the container main body 34. As the porous member 35, for example, a porous material such as a sponge having continuous air bubbles is used, and preferably, a synthetic resin such as polyurethane, polyethylene, or polyvinyl alcohol is used. The porous member 35 is an example of a porous member according to the embodiment of the present invention.

### Actions

The action of the collection container 32 will be described with reference to FIG. 8. The collection container 32 is disposed such that the opening portion 33 thereof is positioned below the power supply brush 15a (not shown) of the power supply brush device 15 in the direction of gravitational force. Even in a case where the size of the abrasion powder D1 generated in association with the sliding contact between the power supply brush 15a and the power supply conductor 9a is large and the slip ring 9 rotates to generate the air flow AF, the abrasion powder D1 often falls in the direction of gravitational force without being scattered. Since the opening portion 33 of the collection container 32 is positioned below the power supply brush 15a in the direction of gravitational force, the collection container 32 can collect the abrasion powder D1 falling in the direction of gravitational force even in a case where the slip ring 9 is rotating.

In addition, in consideration of the fact that the abrasion powder D1 falls in the direction of gravitational force, it is preferable that the power supply brush 15a is disposed below the imaginary horizontal line HL passing through the rotation axis 23 in the direction of gravitational force, that is, in the lower half of the gantry 100.

The signal transmission brush 16a (not shown) of the signal transmission brush device 16 is disposed on the upstream side of the power supply brush 15a of the power supply brush device 15 in the rotation direction R. In addition, the collection container 32 is disposed on the downstream side of the signal transmission brush 16a in the rotation direction R and on the leeward side of the air flow AF as viewed from the signal transmission brush 16a.

The size of the abrasion powder D2 generated in association with the sliding contact between the signal transmission brush 16a and the signal transmission conductor 9b is small. In a case where the slip ring 9 rotates and the air flow AF is generated, the abrasion powder D2 is transported by the air flow AF inside the cover 20 and moves in the rotation direction R. The cover 20 is provided with the collection container 32 having the container main body 34 that extends from the opening portion 33 to a side of the forward direction FW of the air flow AF. Since the abrasion powder D2 transported to the leeward side by the air flow AF moves into the inside of the container main body 34 through the opening portion 33, the abrasion powder D2 can be collected in the collection container 32.

Even in a case where the abrasion powder D1 is scattered, the abrasion powder D1 can be collected in the collection container 32 by the air flow AF.

In addition, since the opening portion 33 of the collection container 32 has a structure that is spread toward the cover 20, the pressure of the air flow AF during the rotation of the slip ring 9 is likely to be lowered. As a result, the scattering of the abrasion powder D2 due to the air flow AF is suppressed. A downward force due to gravity is likely to act on the abrasion powder D2, and the abrasion powder D2 can be induced to the collection container 32, which makes it easy to collect the abrasion powder D2.

According to the embodiment, even during the rotation of the slip ring 9, the two types of the abrasion powders D1 and D2 can be collected by the collection container 32.

Since the porous member 35 is disposed in the collection container 32, the abrasion powders D1 and D2 can be captured. As a result, even in a case where turbulence caused by the air flow AF is generated in the collection container 32, the re-scattering of the abrasion powders D1 and D2 can be prevented.

Even in a case where the collection container 32 does not comprise the porous member 35, the re-scattering of the abrasion powders D1 and D2 can be prevented by increasing the size of the collection container 32 or suctioning the abrasion powders D1 and D2 from a side opposite to the opening portion 33 in the collection container 32.

In addition, it is preferable that the collection container 32 extends in a tangential direction with respect to the outer periphery of the cover 20. In other words, in a case where the tangential direction and the direction (forward direction FW) in which the container main body 34 extends are substantially parallel (including parallel) to each other, the abrasion powder D2 can be effectively collected in the collection container 32 by the air flow AF.

### Modification Example

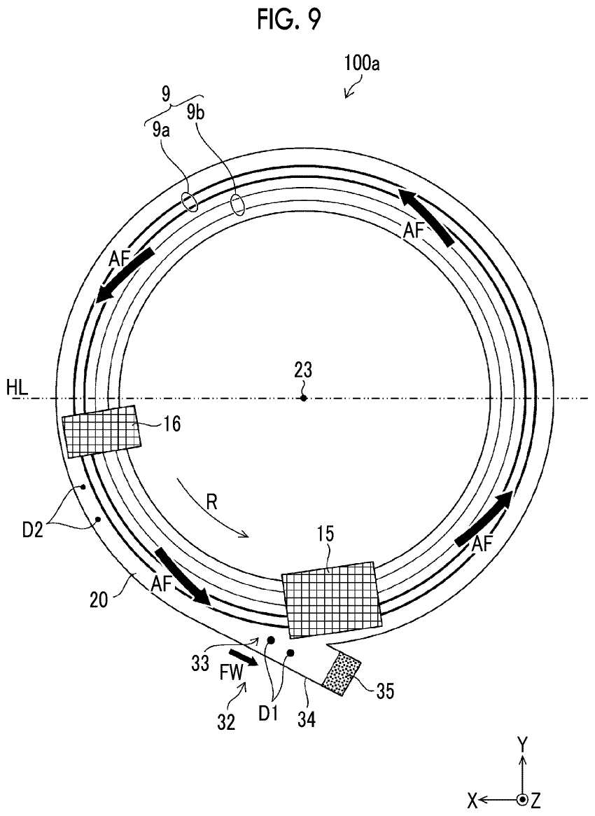

FIG. 9 is a view for describing a modification example of the first embodiment. The same parts as those in the above-described first embodiment are designated by the same reference numerals, and the description thereof will not be repeated.

In the modification example shown in FIG. 9, the positions of the power supply brush device 15 (including the power supply brush 15a), the signal transmission brush device 16 (including the signal transmission brush 16a) (not shown), and the collection container 32 of the present embodiment shown in FIG. 5 are different.

In a gantry 100a of the modification example shown in FIG. 9, the power supply brush device 15, the signal transmission brush device 16, and the collection container 32 are rotated and moved by about 300 in the clockwise direction (direction opposite to the rotation direction R) with respect to the gantry 100 shown in FIG. 5.

Even in the modification example shown in FIG. 9, since the opening portion 33 of the collection container 32 is positioned below the power supply brush device 15 (including the power supply brush 15a) in the direction of gravitational force, the collection container 32 can collect the abrasion powder D1 falling in the direction of gravitational force through the opening portion 33 even in a case where the slip ring 9 is rotating.

In addition, the signal transmission brush device 16 is disposed on the upstream side of the power supply brush device 15 in the rotation direction R, and the collection container 32 is disposed on the leeward side of the air flow AF with respect to the signal transmission brush device 16. Since the container main body 34 of the collection container 32 extends in the forward direction FW of the air flow AF, the abrasion powder D2 transported by the air flow AF can be collected in the collection container 32 through the opening portion 33.

### Second Embodiment

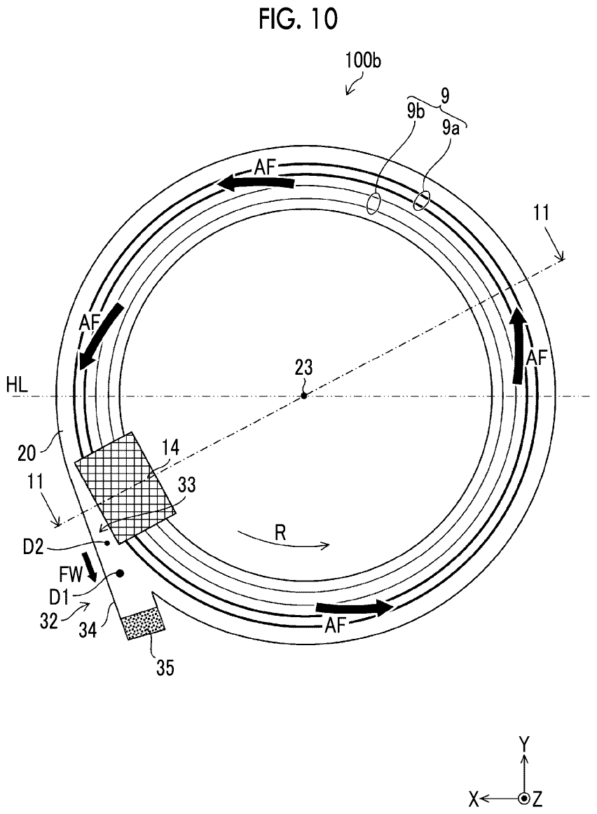

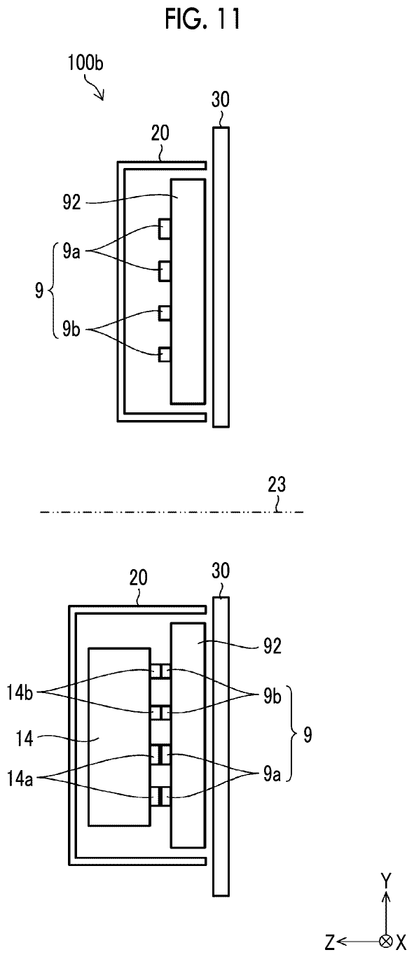

FIGS. 10 and 11 are views for describing a second embodiment. The same parts as those in the above-described first embodiment are designated by the same reference numerals, and the description thereof will not be repeated.

Unlike the first embodiment, a gantry 100b of the second embodiment shown in FIG. 10 comprises one brush device 14. As shown in FIG. 11, the brush device 14 comprises a power supply brush 14a and a signal transmission brush 14b. The power supply brush 14a and the power supply conductor 9a are in contact with each other. In addition, the signal transmission brush 14b and the signal transmission conductor 9b are in contact with each other. As shown in FIGS. 10 and 11, in the second embodiment, the power supply brush 14a and the signal transmission brush 14b are disposed at the same position in the circumferential direction of the slip ring 9. The same position of the power supply brush 14a and the signal transmission brush 14b in the circumferential direction means that the power supply brush 14a and the signal transmission brush 14b are provided in the common brush device 14.

In the second embodiment shown in FIG. 10, the opening portion 33 of the collection container 32 is positioned below the power supply brush 14a of the brush device 14 in the direction of gravitational force. In addition, the container main body 34 of the collection container 32 extends in the forward direction FW of the air flow AF.

As in the first embodiment, the collection container 32 can collect the abrasion powder D1 falling in the direction of gravitational force through the opening portion 33 even in a case where the slip ring 9 is rotating. Since the container main body 34 of the collection container 32 extends in the forward direction FW of the air flow AF, the abrasion powder D2 transported by the air flow AF can be collected in the collection container 32 through the opening portion 33. Even during the rotation of the slip ring 9, the abrasion powders D1 and D2 can be collected in the collection container 32.

In addition, the signal transmission brush 14b is disposed on the upstream side of the collection container 32 in the rotation direction R, and the collection container 32 is disposed on the leeward side of the air flow AF with respect to the signal transmission brush 14b. Since the container main body 34 of the collection container 32 extends in the forward direction FW of the air flow AF, the abrasion powder D2 transported by the air flow AF can be collected in the collection container 32 through the opening portion 33.

Further, it is needless to say that the present invention is not limited to the above-described embodiments and can be variously modified.

### EXPLANATION OF REFERENCES

1: X-ray CT apparatus
15: power supply brush device
15a: power supply brush
16: signal transmission brush device
16a: signal transmission brush
20: cover
23: rotation axis
32: collection container
33: opening portion
34: container main body
35: porous member
D1: abrasion powder
D2: abrasion powder
AF: air flow
FW: forward direction
HL: imaginary horizontal line
R: rotation direction

What is claimed is:

1. An X-ray CT apparatus comprising:
a fixed frame;
a rotation frame that is rotatable with respect to the fixed frame and includes an X-ray source and an X-ray detector;
a slip ring that rotates in synchronization with the rotation frame;
a power supply brush and a signal transmission brush that are in contact with the slip ring;
a cover that covers the slip ring, the power supply brush, and the signal transmission brush; and
a collection container which is provided in the cover and has an opening portion and collects an abrasion powder and in which the opening portion is positioned below the power supply brush in a direction of gravitational force and extends to a side of a forward direction of an air flow occurring due to a rotation of the slip ring.

2. The X-ray CT apparatus according to claim 1,
wherein the opening portion is spread toward the cover.

3. The X-ray CT apparatus according to claim 1,
wherein the collection container extends in a tangential direction with respect to an outer periphery of the cover.

4. The X-ray CT apparatus according to claim 1, further comprising:
a porous member that is disposed inside the collection container.

5. The X-ray CT apparatus according to claim 1,
wherein the power supply brush is positioned below an imaginary horizontal line passing through a rotation axis of the rotation frame in the direction of gravitational force.

6. The X-ray CT apparatus according to claim 5,
wherein the power supply brush and the signal transmission brush are disposed at different positions in a circumferential direction of the slip ring.

7. The X-ray CT apparatus according to claim 6,
wherein the signal transmission brush is disposed on an upstream side of the power supply brush in a rotation direction of the slip ring.

8. The X-ray CT apparatus according to claim 5,
wherein the power supply brush and the signal transmission brush are disposed at the same position in a circumferential direction of the slip ring.

9. The X-ray CT apparatus according to claim 1,
wherein the cover is disposed on a side opposite to the fixed frame with respect to the slip ring.
